(19) United States
(12) Patent Application Publication
(10) Pub. No.: US 2007/0282308 A1
(43) Pub. Date: Dec. 6, 2007

(54) Septum compression rings

(76) Inventor: Benjamin Bell, Haverhill, MA (US)

Correspondence Address: FAY KAPLUN & MARCIN, LLP, 15O BROADWAY, SUITE 702, NEW YORK, NY, 10038, US

(21) Appl. No.: 11/442,908

(22) Filed: May 30, 2006

(51) Int. Cl. A61M 31/00; A61M 39/02; A61M 37/00; A61M 25/16; A61M 25/18

(52) U.S. Cl. 604/539; 604/288.01

(57) ABSTRACT

An access port for a catheter, comprises a housing including a needle opening extending to a fluid chamber defined within the housing and a self sealing septum mounted within the needle opening and engaging an interior perimeter of the needle opening and a compression element disposed between the septum and the housing directing a radially inward compressive force to the septum.

### BACKGROUND

Long term access to the vascular system is often required for the treatment of conditions requiring regular administration of therapeutic agents, nutrition, blood products and/or other fluids or the withdrawal of fluids therefrom. In these cases, a catheter is typically inserted to form a path to the vascular system by advancing a distal end of the catheter into a blood vessel while a proximal end remains accessible.

For example, the proximal end of such a catheter may be connected to a port which may be subcutaneous to minimize interference with patient activities while reducing the probability of infection. In these cases, a needle is often used to pierce the skin and penetrate a self-sealing septum of the port. However, repetitive punctures degrade the ability of the septum to reseal itself, eventually requiring replacement of the port. Extending this “stick life” or “puncture life” of the septum reduces the number of these replacement procedures consequently reducing the discomfort and cost associated with the procedures.

### SUMMARY OF THE INVENTION

The present invention is directed to an access port for a catheter, comprising a housing including a needle opening extending to a fluid chamber defined within the housing and a self sealing septum mounted within the needle opening and engaging an interior perimeter of the needle opening in combination with a compression element disposed between the septum and the housing directing a radially inward compressive force to the septum.

### BRIEF DESCRIPTION OF DRAWINGS

FIG. 1 is an exploded view showing an access port according to an embodiment of the present invention;

FIG. 2 is a cross sectional view of the access port shown in FIG. 1, and

FIG. 3 is a cross sectional view of another embodiment of the access port according to the invention.

### DETAILED DESCRIPTION

The present invention may be further understood with reference to the following description and to the appended drawings, wherein like elements are referred to with the same reference numerals. The present invention relates to devices for accessing the vascular system via a catheter and, more specifically, relates to an access port for the injection and/or withdrawal of fluids via a subcutaneous central venous access catheter.

The present invention provides a method and system increasing the puncture life of the septum by applying a radially compressive force to the septum. In one exemplary embodiment, septum compression rings are provided to generate the radial compression, while minimizing the resulting axial force that may be applied to the housing assembly and to joints and bonds of the port. For example, the force may result from the septum compression rings providing an interference fit between the septum and a housing of the port. The septum compression rings may be formed, for example, by beads of additional septum material disposed around an outer diameter of the septum.

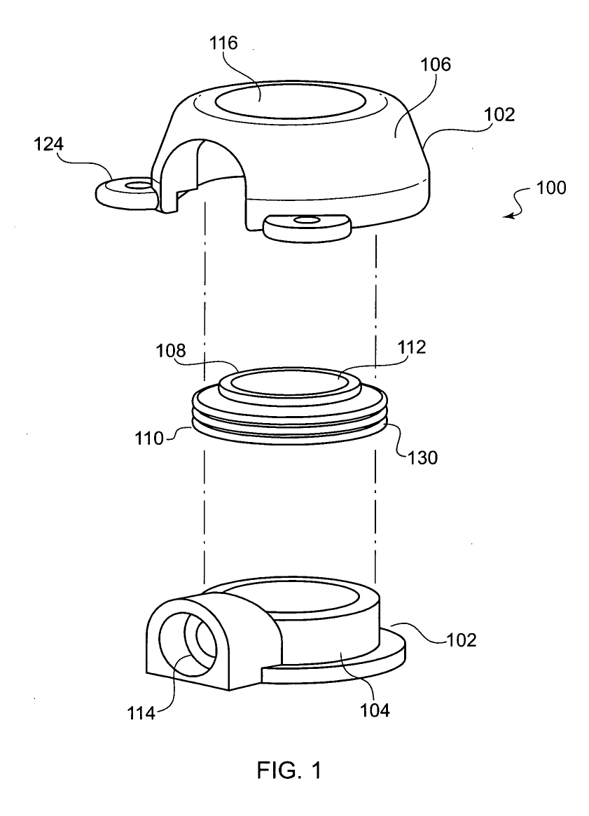

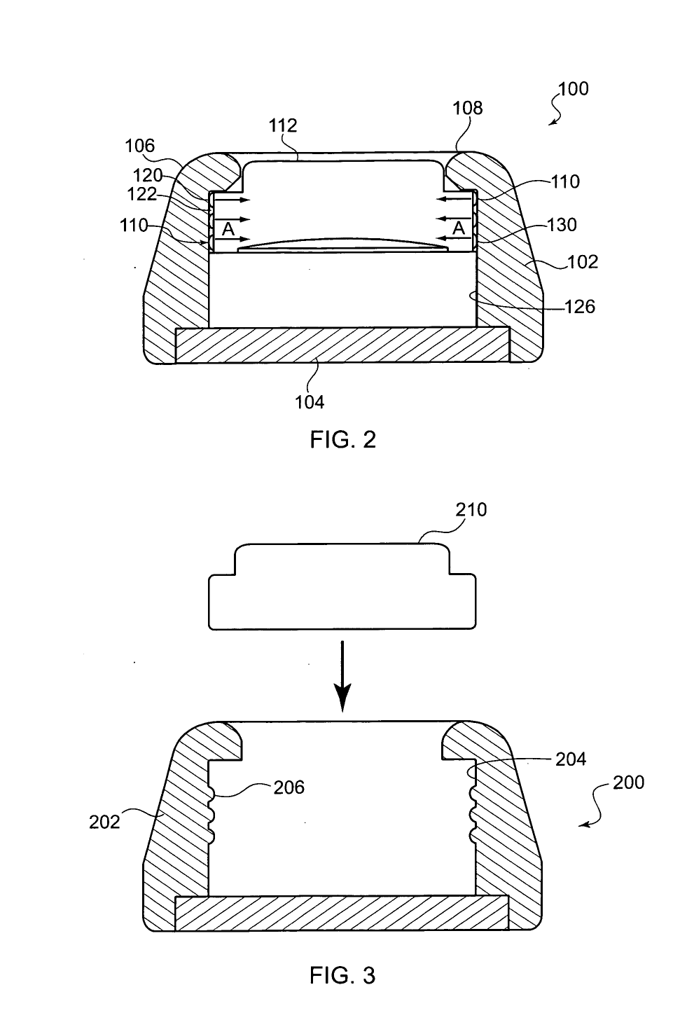

As shown in FIGS. 1 and 2, an access port 100 which may, for example, be a subcutaneous venous access port connected to an implanted catheter via a connector 114. The port 100 comprises a housing 102 including a base 104 and an upper portion 106 which defines an internal fluid chamber 105 in fluid connection with an outlet/inlet 113 in the connector 114 for fluid connection to a catheter. The base 104 and upper portion 106 may be joined to one another by, for example, a friction fit, mechanical interlocks, by bonding or with an adhesive, among other options. The housing 102 may further comprise suture loops 124 or similar elements to secure the device to tissue. A needle opening 116 is provided on a side of the port 100 facing the underside of the skin so that a needle inserted through the skin can pass through the opening 116 into the chamber 105 to fluidly couple to the catheter and, consequently, to the vascular system.

The port 100 comprises a septum 108 held in place between the base 102 and upper portion 106. For example, a perimeter of the septum 108 may be held between opposing surfaces of the base 104 and upper portion 106. A compression ring element 110 is formed by one or more rings 130 disposed on the outer diameter of the septum 108. The compression ring element 110 is placed in radial interference with inner surfaces 126 of the upper portion 106 of the housing 102. The radial interference causes a substantially radial force A that compresses the membrane 112 of the septum 108. The force has a minimal axial component, so that only a small axial force is applied to the joints and bonds of the housing 102 during use.

According to the invention, the size, shape and number of the individual rings or beads 130 forming the compression ring element 110 may be selected to tailor the forces applied to the housing 102 and the septum 108 to a desired level or to position the forces in a desired manner. For example, the friction between the compression ring element 110 and the upper portion 106 may be reduced by reducing the surface area of the beads 130 contacting the surface 126 during assembly of the device, such as when the septum 108 is pushed in place.

In one exemplary embodiment, the compression ring element 110 comprises three rings or beads 130 having a substantially semi-circular cross section. As shown in FIGS. 1 and 2, the beads 130 form peaks 120 in interference contact with the inner surface 126 of the housing 102 to generate the compressive force. Troughs 122 are formed between the peaks 120 reducing the surface area of the compressive ring element 110 in contact with the inner surface 126 of the upper portion 106. The reduced contact area lessens the friction that must be overcome when inserting the septum 108 in the housing 102, and also lessens the axial force applied to the assembly.

A desired radial compression of the septum 108 may be achieved by selecting an appropriate number, shape and location of the rings or beads 130. For example, the amount of compression and the distribution of the compressive force on the membrane 112 may be modified by selecting different parameters of the compression ring element 110. The geometry of the individual beads 130 may be changed to achieve a desired compressive force and friction with the housing. For example, in addition to the semi-circular cross section of the beads 130 shown in the drawings, different geometries may be used such as triangular, trapezoidal or block shaped beads. These parameters may be varied as necessary to normalize the septum compression distribution across the volume of the septum.

In a different embodiment, the compression force on the membrane 112 may be achieved without the addition of specific rings or beads. Instead, in this embodiment an oversized diameter of the septum perimeter creates the interference with the housing of the port and the resulting radial compression of the septum. The oversized septum perimeter may have, for example, a diameter slightly larger than an inner diameter of the opening in the housing component within which it is to reside.

In yet another embodiment, the compression ring features may be applied to the housing of the port rather than to the septum. As shown in FIG. 3, a port 200 according to a further embodiment of the invention includes a housing formed of an upper portion 202 defining an opening 207 within which a septum 210 resides. Beads 206 formed on an inner surface 204 of the upper portion 202 form radial compression elements causing interference with the perimeter of the septum 210 and the resulting radially compressive force applied thereto.

According to the exemplary embodiments of the invention, the compression rings may be formed of the same material as that of which the septum is formed. For example, the septum and/or the compression rings may be formed of silicone or any of a variety of materials of different durometer values.

The present invention has been described with reference to specific exemplary embodiments. Those skilled in the art will understand that changes may be made in details, particularly in matters of shape, size, material and arrangement of parts. Accordingly, various modifications and changes may be made to the embodiments. The specifications and drawings are, therefore, to be regarded in an illustrative rather than a restrictive sense.

What is claimed is:

1. An access port for a catheter, comprising:
a housing including a needle opening extending to a fluid chamber defined within the housing;
a self sealing septum mounted within the needle opening and engaging an interior perimeter of the needle opening; and
a compression element disposed between the septum and the housing directing a radially inward compressive force to the septum.

2. The port according to claim 1, wherein the compression element is formed integrally with the septum.

3. The port according to claim 1, wherein the compression element is formed on the interior perimeter of the needle opening.

4. The port according to claim 1, wherein the compression element is formed on a surface of the septum engaging the interior perimeter of the needle opening.

5. The port according to claim 1, wherein the compression element comprises at least one bead extending around a perimeter of the needle opening.

6. The port according to claim 5, wherein the septum and the bead are formed of the same material.

7. The port according to claim 5, wherein the compression element comprises three beads.

8. The port according to claim 5, wherein the a cross section of the at least one bead is shaped as one of a semi-circle, a triangle, a trapezoid and a block.

9. The port according to claim 1, wherein an outer diameter of a portion of the septum engaging the interior perimeter of the needle opening is larger than a diameter of the interior perimeter of the needle opening and the compression element is comprised of a portion of the septum which, in an uncompressed state, extends radially beyond a diameter of the interior perimeter of the needle opening.

10. The port according to claim 1, wherein a durometer of a material of which the compression element is formed is different than a durometer of a material of which a portion of the septum extending over the needle opening is formed.

11. The port according to claim 1, wherein the compression element comprises alternating troughs and peaks in contact with the interior perimeter of the needle opening.

12. A septum for a subcutaneous access port, comprising:
a self sealing septum extending across a needle opening of the access port with an outer surface of the septum facing an inner diameter of the needle opening; and
a compression element disposed between the outer surface of the septum and the inner diameter of the needle opening, the compression element applying a substantially radially inwardly directed compressive force to the septum.

13. The septum according to claim 12, wherein the compression element is formed as at least one bead of material extending around at least a portion of a diameter of the needle opening, the at least one bead of material being bonded to one of the outer surface of the septum and the interior perimeter of the needle opening.

14. The septum according to claim 13, wherein the a cross section of the at least one bead is shaped as one of a semi-circle, a triangle, a trapezoid and a block.

15. The septum according to claim 12, wherein the septum is made of silicone.

16. The septum according to claim 12, wherein a durometer of the compression element is different from a durometer of the septum.

17. The septum according to claim 12, wherein the compression element comprises an oversized perimeter of the septum.

18. The septum according to claim 13, the at least one bead comprises a plurality of beads, a first one of the beads having dimensions different than those of a second one of the beads to generate different compressive forces in different portions of the septum.

19. The septum according to claim 18, wherein the plurality of beads defines peaks and troughs of the compression element, the peaks engaging the interior perimeter of the needle opening.

20. The septum according to claim 18, wherein the plurality of beads comprises three beads.

21. The septum according to claim 12, wherein the compression element is placed in radial interference with the interior perimeter of the needle opening.

22. The septum according to claim 12, wherein the compression element and the septum are integrally formed.

23. The septum according to claim 12, wherein the septum and the compression element are formed of substantially the same material.
